(19) United States
(12) Patent Application Publication
(10) Pub. No.: US 2023/0245727 A1
(43) Pub. Date: Aug. 3, 2023

(54) METHOD FOR MOLECULAR REPRESENTING

(71) Applicant: BEIJING BAIDU NETCOM SCIENCE TECHNOLOGY CO., LTD., Beijing (CN)

(72) Inventors: Donglong HE, Beijing (CN); Lihang Liu, Beijing (CN); Dayong Lin, Beijing (CN); Xiaomin Fang, Beijing (CN); Fan Wang, Beijing (CN); Jingzhou He, Beijing (CN)

(73) Assignee: BEIJING BAIDU NETCOM SCIENCE TECHNOLOGY CO., LTD., Beijing (CN)

(21) Appl. No.: 18/126,887

(22) Filed: Mar. 27, 2023

(30) Foreign Application Priority Data
Mar. 28, 2022 (CN) 202210314863.0

(51) Int. Cl. G16C 20/30; G16C 20/50; G16C 20/70

(52) U.S. Cl. CPC G16C 20/30; G16C 20/50; G16C 20/70

(57) ABSTRACT

A computer-implemented method is provided. The method includes: obtaining feature information of a molecule to be represented, wherein the molecule includes a plurality of atoms; generating a fully connected graph of the plurality of atoms, wherein the fully connected graph includes a plurality of edges; generating, based on the feature information, a plurality of atom vector representations and a plurality of edge vector representations, wherein the plurality of atom vector representations correspond to the plurality of atoms, respectively, and the plurality of edge vector representations correspond to the plurality of edges, respectively; performing, based on the fully connected graph, at least one aggregation on the plurality of atom vector representations and the plurality of edge vector representations to obtain a plurality of updated atom vector representations; and generating, based on the plurality of updated atom vector representations, a molecular vector representation of the molecule.

### CROSS REFERENCE TO RELATED APPLICATION

This application claims priority to Chinese patent application No. 202210314863.0 filed on Mar. 28, 2022, the contents of which are hereby incorporated by reference in their entirety for all purposes.

### TECHNICAL FIELD

The present disclosure relates to the technical field of artificial intelligence, in particular to the technical field of biological computing and deep learning, and in particular to a molecular representation method and apparatus, a method and apparatus for training a molecular representation model, an electronic device, a computer-readable storage medium, and a computer program product.

### BACKGROUND

Artificial Intelligence (AI) is a discipline that studies how to make computers simulate certain thinking processes and intelligent behaviors (such as learning, reasoning, thinking, and planning) of human beings. AI has both hardware technology and software technology. The hardware technology of artificial intelligence generally includes sensors, dedicated artificial intelligence chips, cloud computing, distributed storage, big data processing, etc. The software technology of artificial intelligence mainly includes computer vision technology, speech recognition technology, natural language processing technology, machine learning/deep learning, big data processing technology, knowledge graph technology and other major directions.

In recent years, AI-driven drug design has attracted more and more attention. The deep learning technology is used to predict the attributes of drug molecules, such as drug toxicity, stability, and affinity of drug ligands to protein receptors.

Methods described in this section are not necessarily those previously envisaged or adopted. Unless otherwise specified, it should not be assumed that any method described in this section is considered the prior art only because it is included in this section. Similarly, unless otherwise specified, the issues raised in this section should not be considered to have been universally acknowledged in any prior art.

### SUMMARY

The present disclosure provides a method for molecular representing, an electronic device, and a computer-readable storage medium.

According to one aspect of the present disclosure, a computer-implemented method is provided, and includes: obtaining a feature information of a molecule to be represented, wherein the molecule comprises a plurality of atoms; generating a fully connected graph of the plurality of atoms, wherein the fully connected graph comprises a plurality of edges; generating, based on the feature information, a plurality of atom vector representations and a plurality of edge vector representations, wherein the plurality of atom vector representations correspond to the plurality of atoms respectively, and the plurality of edge vector representations correspond to the plurality of edges respectively; performing, based on the fully connected graph, at least one aggregation on the plurality of atom vector representations and the plurality of edge vector representations to obtain a plurality of updated atom vector representations; and generating, based on the plurality of updated atom vector representations, a molecular vector representation of the molecule.

According to one aspect of the present disclosure, an electronic device, comprising: one or more processors; and a memory storing one or more programs configured to be executed by the one or more processors, the one or more programs comprising instructions for performing operations comprising: obtaining a feature information of a molecule to be represented, wherein the molecule comprises a plurality of atoms; generating a fully connected graph of the plurality of atoms, wherein the fully connected graph comprises a plurality of edges; generating, based on the feature information, a plurality of atom vector representations and a plurality of edge vector representations, wherein the plurality of atom vector representations correspond to the plurality of atoms respectively, and the plurality of edge vector representations correspond to the plurality of edges respectively; performing, based on the fully connected graph, at least one aggregation on the plurality of atom vector representations and the plurality of edge vector representations to obtain a plurality of updated atom vector representations; and generating, based on the plurality of updated atom vector representations, a molecular vector representation of the molecule.

According to one aspect of the present disclosure, a non-transitory computer-readable storage medium storing one or more programs comprising instructions that, when executed by one or more processors of a computing device, cause the computing device to perform operations comprising: obtaining a feature information of a molecule to be represented, wherein the molecule comprises a plurality of atoms; generating a fully connected graph of the plurality of atoms, wherein the fully connected graph comprises a plurality of edges; generating, based on the feature information, a plurality of atom vector representations and a plurality of edge vector representations, wherein the plurality of atom vector representations correspond to the plurality of atoms respectively, and the plurality of edge vector representations correspond to the plurality of edges respectively; performing, based on the fully connected graph, at least one aggregation on the plurality of atom vector representations and the plurality of edge vector representations to obtain a plurality of updated atom vector representations; and generating, based on the plurality of updated atom vector representations, a molecular vector representation of the molecule.

It should be understood that the content described in this part is not intended to identify key or important features of the embodiments of the present disclosure, nor is it used to limit the scope of the present disclosure. Other features of the present disclosure will be easily understood by the following description.

### BRIEF DESCRIPTIONS OF THE DRAWINGS

The accompanying drawings illustrate the embodiments by way of example and constitute a part of the specification, and together with the written description of the specification serve to explain example implementations of the embodiments. The illustrated embodiments are for illustrative purposes only and do not limit the scope of the claims. Throughout the drawings, the same reference numerals refer to similar but not necessarily identical elements.

FIG. 1 shows a flow diagram of a molecular representation method according to some embodiments of the present disclosure;

FIG. 2 shows a schematic diagram of a fully connected graph of atoms according to some embodiments of the present disclosure;

FIG. 3 shows a schematic diagram of updating atom vector representations according to some embodiments of the present disclosure;

FIG. 4 shows a schematic diagram of updating edge vector representations according to some embodiments of the present disclosure;

FIG. 5 shows a schematic diagram of an edge vector representation aggregation based on adjacent edge pairs according to some embodiments of the present disclosure;

FIG. 6 shows a flow diagram of a method for training a molecular representation model according to some embodiments of the present disclosure;

FIG. 7 shows a schematic diagram of a training process of a molecular representation model according to some embodiments of the present disclosure;

FIG. 8 shows a structural block diagram of a molecular representation model according to some embodiments of the present disclosure;

FIG. 9 shows a structural block diagram of an aggregation updating module according to some embodiments of the present disclosure;

FIG. 10 shows a schematic diagram of a node-edge attention mechanism according to some embodiments of the present disclosure;

FIG. 11 shows a schematic diagram of an edge attention mechanism according to some embodiments of the present disclosure;

FIG. 12 shows a structural block diagram of a molecular representation apparatus according to an embodiment of the present disclosure;

FIG. 13 shows a structural block diagram of an apparatus for training a molecular representation model according to an embodiment of the present disclosure; and

FIG. 14 shows a structural block diagram of an example electronic device that can be configured to implement embodiments of the present disclosure.

### DETAILED DESCRIPTION OF THE EMBODIMENTS

The embodiments of the present disclosure are described below with reference to the accompanying drawings, including various details of the embodiments of the present disclosure to facilitate understanding, which should be considered merely example. Therefore, those of ordinary skill in the art should recognize that various changes and modifications may be made to the embodiments described herein without departing from the scope of the present disclosure. Similarly, for clarity and conciseness, the description of well-known functions and structures is omitted from the following description.

In the present disclosure, unless otherwise specified, the terms “first”, “second” and the like are used to describe various elements and are not intended to limit the positional relationship, temporal relationship or importance relationship of these elements. These terms are only used to distinguish one element from another element. In some examples, a first element and a second element may refer to the same instance of the element, and in some cases, based on the context description, they can also refer to different instances.

The terms used in the description of the various examples in the present disclosure are only for the purpose of describing specific examples and are not intended to be limiting. Unless the context clearly indicates otherwise, if the number of elements is not specifically limited, the element may be one or more. In addition, the term “and/or” as used in the present disclosure covers any and all possible combinations of the listed items.

In the present disclosure, treatments such as collection, storage, use, processing, transmission, provision, and disclosure of involved personal information of the user are all in compliance with relevant laws and regulations, and do not violate public order and good customs.

In recent years, AI-driven drug design has attracted more and more attention. The deep learning technology is used to predict the attributes of drug molecules, such as drug toxicity, stability, and affinity of drug ligands to protein receptors. High-quality molecular representations can improve the accuracy of molecular attribute prediction, greatly improve the efficiency of drug development, and reduce costs.

Therefore, an embodiment of the present disclosure provides a molecular representation method that can obtain a high-quality molecular vector representation, thereby improving the accuracy of molecular attribute prediction.

The embodiment of the present disclosure will be described in detail below with reference to the accompanying drawings.

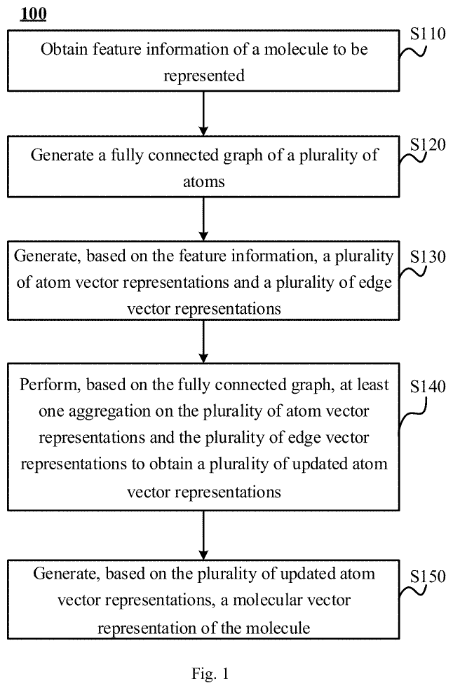

FIG. 1 shows a flowchart of the molecular representing method 100 according to the embodiment of the present disclosure. The method 100 may be executed at a server or at a client device. That is, an execution body of each step of the method 100 may be the server or the client device.

As shown in FIG. 1, the method 100 includes S110-S150.

In step S110, feature information of a molecule to be represented is obtained. The molecule includes a plurality of atoms.

In step S120, a fully connected graph of the plurality of atoms is generated. The fully connected graph includes a plurality of edges.

In step S130, a plurality of atom vector representations and a plurality of edge vector representations are generated based on the feature information. The plurality of atom vector representations correspond to the plurality of atoms, respectively. The plurality of edge vector representations correspond to the plurality of edges, respectively.

In step S140, at least one aggregation is performed on the plurality of atom vector representations and the plurality of edge vector representations based on the fully connected graph to obtain a plurality of updated atom vector representations.

In step S150, a molecular vector representation of the molecule is generated based on the plurality of updated atom vector representations.

Attributes of the molecule are essentially a result of interaction between the atoms, and edges between the atoms can express the connectivity and interaction between the atoms. According to the embodiment of the present disclosure, by constructing the fully connected graph of the atoms and performing aggregation on the atom vector representations and the edge vector representations, atom information and edge information can be fully interacted, thereby obtaining the more comprehensive and accurate molecular vector representation.

The molecular vector representation of the embodiment of the present disclosure can fully and accurately express the properties of the molecule. Further, by predicting the attributes of the molecules according to the molecular vector representation of the embodiment of the present disclosure, the accuracy of molecular attribute prediction can be improved, thereby greatly improving the efficiency of drug research and development.

The molecular representing method of the embodiment of the present disclosure is suitable for processing a molecule including a plurality of atoms and a plurality of chemical bonds.

In the embodiment of the present disclosure, the fully connected graph of the plurality of atoms may be constructed based on the plurality of atoms included in the molecule, wherein the plurality of atoms of the molecular correspond to a plurality of nodes in the fully connected graph. In the fully connected graph, any two atoms are connected by an edge. It can be understood that the number of the edges included in the fully connected graph is N(N−1)/2, where N is the number of atoms.

The plurality of edges of the fully connected graph at least include the plurality of chemical bonds in the molecule. In the case where each pair of atoms in the molecule are connected via the chemical bonds, the plurality of edges of the fully connected graph are all chemical bonds. When there are atom pairs that are not connected via the chemical bonds in the molecule, the plurality of edges of the fully connected graph include not only the chemical bonds in the molecule, but also virtual edges between every atom pair that are not connected via the chemical bonds. It should be understood that in the embodiment of the present disclosure, the virtual edges refer to any edge except the plurality of chemical bonds among the plurality of edges included in the fully connected graph.

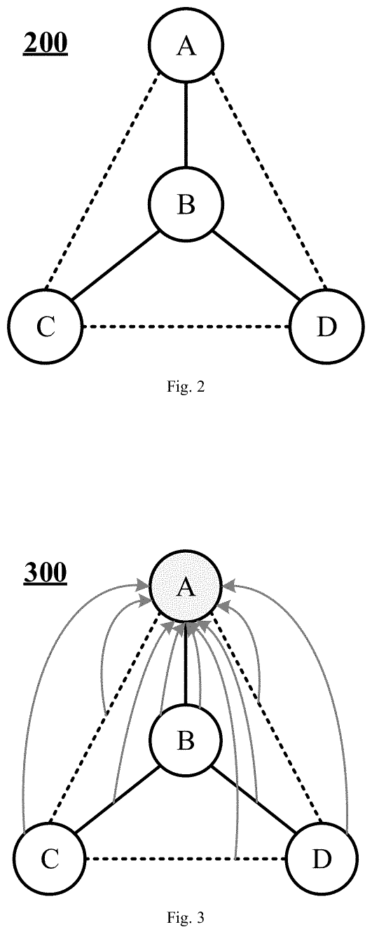

FIG. 2 shows a schematic diagram of the fully connected graph of the atoms according to the embodiment of the present disclosure. As shown in FIG. 2, the molecule 200 includes four atoms A, B, C, and D, and three chemical bonds AB, BC, and BD between corresponding two atoms. In FIG. 2, the chemical bonds are represented by solid lines. By constructing the fully connected graph, a virtual edge AC is added between the atom A and the atom C, a virtual edge AD is added between the atom A and the atom D, and a virtual edge CD is added between the atom C and the atom D. In FIG. 2, the virtual edges are represented by dashed lines.

For step S130, according to some embodiments, the feature information of the molecule includes atom feature information of each of the plurality of atoms and chemical bond feature information of each of the plurality of chemical bonds.

The atom feature information includes, for example, the serial number of each atom, spatial coordinates, hybridization manner, degree (that is, the number of connected atoms), the number of connected hydrogen atoms, valence, whether the atoms are in an aromatic system, and whether the atoms are in a loop.

The chemical bond feature information includes, for example, the type of the chemical bonds, stereoisomerism, bond length, bond angle, whether the chemical bonds are aromatic bonds, and whether the chemical bonds are in a loop.

According to some embodiments, the feature information of the molecule may be obtained by analyzing molecular description data such as a simplified molecular input line entry specification (SMILES) expression of the molecule and a structure data file (SDF) chemical data file. According to other embodiments, the feature information of the molecule may also be obtained by using an open-source toolkit for cheminformatics such as RDKit.

The atom vector representation of each atom and the edge vector representation of each edge may be generated based on the feature information of the molecule. Specifically, the atom vector representation of each atom may be generated at least based on the corresponding atom feature information. The edge vector representation of each chemical bond may be generated at least based on the corresponding chemical bond feature information. In the case where the fully connected graph includes the virtual edges (that is, in the case where the number of the plurality of edges included in the fully connected graph is greater than the number of the plurality of chemical bonds), edge vector representations of the virtual edges may be set to a preset value.

According to some embodiments, the atom vector representation of any atom may be generated by encoding the atom feature information of the atom. According to other embodiments, the atom vector representation of any atom may be generated by encoding the atom feature information of the atom and the chemical bond feature information of the chemical bond to which the atom is connected.

According to some embodiments, the edge vector representation of any chemical bond may be generated by encoding the chemical bond feature information of the chemical bond. According to other embodiments, the edge vector representation of any chemical bond may be generated by encoding the chemical bond feature information of the chemical bond and the atom feature information of the atoms to which the chemical bond is connected.

In the case where the fully connected graph includes the virtual edges, the edge vector representations of the virtual edges may be set to a preset value, such as an all-zero vector.

According to some embodiments, the atom vector representations have the same dimension (for example, 100 dimensions) as the edge vector representations, so that the computational efficiency of subsequent steps can be improved.

It should be understood that the atom vector representations and the edge vector representations generated based on the feature information of the molecule are both initial values. In the subsequent step S140, at least one iteration updating is performed on each of the atom vector representations and the edge vector representations.

According to some embodiments, in step S140, at least one aggregation is performed on the plurality of atom vector representations and the plurality of edge vector representations based on the fully connected graph, and after each aggregation, values of the plurality of atom vector representations and the plurality of edge vector representations are updated, so that the plurality of updated atom vector representations and a plurality of updated edge vector representations are obtained.

According to some embodiments, each aggregation of the at least one polymerization includes the following steps S142-S146.

In step S142, the aggregation is performed on the plurality of current atom vector representations and the plurality of current edge vector representations based on an attention mechanism to obtain the updated atom vector representation of any atom of the plurality of atoms.

In step S144, a current edge vector representation of the edge is updated based on updated atom vector representations of two atoms connected by the edge to obtain a first edge vector representation of any edge of the plurality of edges.

In step S146, the aggregation is performed on the plurality of first edge vector representations of the plurality of edges based on the attention mechanism to obtain the updated edge vector representation of any edge of the plurality of edges.

According to the above embodiment, in each aggregation process, first, the atom vector representations are updated by performing the aggregation on each atom vector representation and each edge vector representation (step S142 of the current aggregation). Then, the updated atom vector representations are transferred to the edge vector representations (step S144 of the current aggregation). Finally, the edge vector representations are updated by performing the aggregation on the edge vector representations (step S146 of the current aggregation). The updated edge vector representations may be used to update the atom vector representations in the next aggregation (step S142 of the next aggregation). In this way, full interaction of atom information and edge information can be achieved, and each atom vector representation and each edge vector representation can learn more comprehensive and accurate information, thereby improving the accuracy of the final molecular vector representation.

FIG. 3 shows a schematic diagram of updating the atom vector representations according to some embodiments of the present disclosure. The process shown in FIG. 3 corresponds to the above step S142.

As shown in FIG. 3, a molecule 300 includes four atoms A, B, C, and D. Taking the atom A as an example, aggregation (an information aggregation direction is as shown by gray arrows in the figure) is performed on atom vector representations of the atoms A, B, C, and D and edge vector representations of edges AB, AC, AD, BC, BD, and CD based on the attention mechanism, so that an updated atom vector representation of the atom A may be obtained. In the aggregation process, an attention weight of each vector representation (including the atom vector representations and the edge vector representations) may be obtained in advance by training.

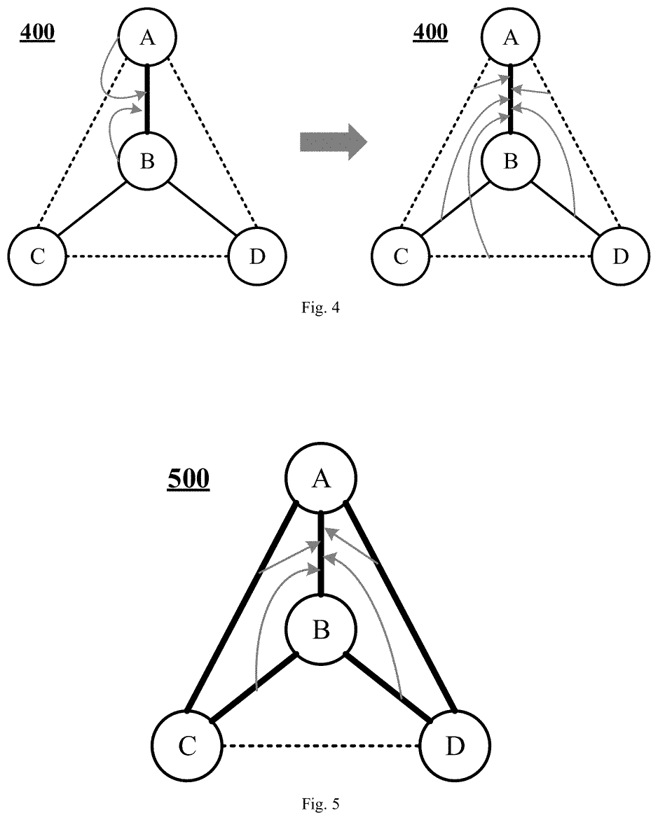

FIG. 4 shows a schematic diagram of updating the edge vector representations according to some embodiments of the present disclosure. The process shown in FIG. 4 corresponds to the above steps S144 and S146.

As shown in FIG. 4, a molecule 400 includes six edges: AB, AC, AD, BC, BD, and CD. Taking the edge AB as an example, first, a vector representation of the edge AB is updated (an information updating direction is as shown by gray arrows in the left figure) based on the updated atom vector representations of the atom A and the atom B, so as to obtain a first edge vector representation of the edge AB. First edge vector representations of the other five edges, that is, AC, AD, BC, BD, and CD, may also be obtained in a similar manner. Then, still taking the edge AB as an example, an aggregation (an information aggregation direction is as shown by gray arrows in the right figure) is performed on the first edge vector representation of the edge AB and the first edge vector representations of the other five edges based on the attention mechanism, so as to obtain an updated edge vector representation of the edge AB. In the aggregation process, the attention weight of each edge vector representation may be obtained in advance by training.

According to some embodiments, the above step S144, that is, the current edge vector representation of the edge is updated based on the updated atom vector representations of the two atoms connected by the edge to obtain the first edge vector representation of the edge, further includes the following steps S1442 and S1444.

Step S1442, a vector representation variation of the edge is determined based on the updated atom vector representations of the two atoms connected by the edge; and

Step S1444, the current edge vector representation of the edge and the vector representation variation are added to obtain the first edge vector representation of the edge.

According to the above embodiment, the updated atom vector representations may be transferred to the edge vector representations, thereby realizing the supplementation and augmentation of the edge information.

According to some embodiments, for the above step S1442, a matrix may be obtained by calculating an outer product of the updated atom vector representations of the two atoms, and then the matrix is dimensionally reduced to be a vector by means of averaging, linear transformation, etc. The vector is the vector representation variation of the corresponding edge. Then, for step S1444, the current edge vector representation of the edge and the vector representation variation of the edge are added to obtain the first edge vector representation of the edge.

According to some embodiments, the above step S146, that is, the aggregation is performed on the plurality of first edge vector representations of the plurality of edges based on the attention mechanism to obtain the updated edge vector representation of the edge, further includes the following steps S1462 and S1464:

S1462, at least one adjacent edge pair of the edge is determined, where each adjacent edge pair of the at least one adjacent edge pair includes two adjacent edges of the edge, and the two adjacent edges are connected with the edge to form a triangle; and

S1464, the aggregation is performed on the edge and a first edge vector representation of each adjacent edge in the at least one adjacent edge pair based on the attention mechanism to obtain the updated edge vector representation of the edge.

The three edges of the triangle are constrained to one another, and the properties of one edge are greatly influenced by adjacent edges of the triangle. According to the above embodiment, when the edge vector representation of an edge is updated by aggregation, only the edge vector representation of the adjacent edges that have a triangular relationship with the edge are aggregated, which can greatly reduce the amount of calculation (compared to aggregation on the edge vector representations of all the edges) and improve computational efficiency on the premise of ensuring that key information is not omitted.

FIG. 5 shows a schematic diagram of an edge vector representation aggregation based on adjacent edge pairs according to some embodiments of the present disclosure. As shown in FIG. 5, a molecule 500 includes six edges: AB, AC, AD, BC, BD, and CD. Taking the edge AB as an example, the edge includes two adjacent edge pairs, namely (AC, BC) and (AD, BD). Correspondingly, the aggregation (the information aggregation direction is as shown by gray arrows in the figure) may be performed on the first edge vector representations of the edge AB and each adjacent edge in the adjacent edge pair, that is, the edges AC, BC, AD, and BD, based on the attention mechanism, so as to obtain the updated edge vector representation of the edge AB.

According to some embodiments, two adjacent edges of each adjacent edge pair include a first adjacent edge connected to a first end point of the edge (also referred to as a “start point” of the edge) and a second adjacent edge connected to a second end point of the edge (also referred to as a “terminal point” of the edge). Correspondingly, the above step S1464, that is, the aggregation is performed on the edge and the first edge vector representation of each adjacent edge in the at least one adjacent edge pair based on the attention mechanism to obtain the updated edge vector representation of the edge, further includes the following steps S14642 and S14644.

S14642, aggregation is performed on the edge and a first edge vector representation of each first adjacent edge in the at least one adjacent edge pair based on the attention mechanism to obtain a second edge vector representation of the edge; and

S14644, aggregation is performed on the edge and a second edge vector representation of each second adjacent edge in the at least one adjacent edge pair based on the attention mechanism to obtain the updated edge vector representation of the edge.

According to the above embodiment, aggregation is performed first on the first adjacent edges connected to the first end point, and then on the second adjacent edges connected to the second endpoint, so that sufficient information interaction between the edges can be realized.

According to some embodiments, in an edge attention mechanism of the above steps S1464, S14642, and S14644, attention weights of the edge and each adjacent edge in the at least one adjacent edge pair are determined at least based on the shortest chemical bond distance between the corresponding two atoms. Thus, chemical bond distance information between atoms can be introduced into the process of edge information aggregation, the updated edge vector representations integrate spatial structure information of the molecule, and the more comprehensive and accurate molecular vector representation can be obtained.

The shortest chemical bond distance refers to the number of chemical bonds included in the shortest chemical bond path connecting two atoms. According to some embodiments, the weights of the edge and each adjacent edge may be obtained in advance by training based on the shortest chemical bond distance between the corresponding two atoms.

Through the at least one aggregation in step S140, the plurality of updated atom vector representations and the plurality of updated edge vector representations may be obtained.

Then, in step S150, the molecular vector representation of the molecule may be generated based on the plurality of updated atom vector representations.

There are several ways to generate the molecular vector representation based on the plurality of atom vector representations.

According to some embodiments, the molecular vector representation may be obtained by concatenating the plurality of atom vector representations.

According to other embodiments, the molecular vector representation may be obtained by adding elements of corresponding positions of a plurality of atom vectors.

According to other embodiments, a weighted summation result of the plurality of atom vectors may be represented as the molecular vector representation.

According to other embodiments, the plurality of atom vectors may be input into a trained multi-layer perceptron (MLP), and output of the MLP may be represented as the molecular vector representation.

The molecular vector representation obtained in step S150 may be used to predict the attributes of the molecule. That is, according to some embodiments, the method 100 further includes: the attributes of the molecule are predicted based on the molecular vector representation.

Since the molecular vector representation generated according to the embodiment of the present disclosure can comprehensively and accurately express the properties of the molecule, by predicting the attributes of the molecules based on the molecular vector representation of the embodiment of the present disclosure, the accuracy of molecular attribute prediction can be improved, thereby greatly improving the efficiency of drug research and development.

According to some embodiments, the attributes of the molecule may include at least one of: water solubility, toxicity, degree of matching with preset proteins, compound reactivity, stability, degradability, and energy.

According to some embodiments, the molecular vector representation may be input into a predictor to obtain the attributes, output by the predictor, of the molecule. The predictor may be, for example, a feed forward neural network.

According to some embodiments, the above steps S140 and S150 may be implemented by the trained molecular representation model. According to some embodiments, the molecular vector representation output by the molecular representation model may be obtained by inputting the fully connected graph, the plurality of atom vector representations and the plurality of edge vector representations into the trained molecular representation model.

According to some embodiments, the trained molecular representation model may include an aggregation updating module and a representation module. Correspondingly, step S140 may further include: the fully connected graph, the plurality of atom vector representations and the plurality of edge vector representations are input into the aggregation updating module of the trained molecular representation model to obtain the plurality of updated atom vector representations output by the aggregation updating module. Step S150 may further include: the plurality of updated atom vector representations are input into the representation module of the molecular representation model to obtain the molecular vector representation, output by the representation module, of the molecule.

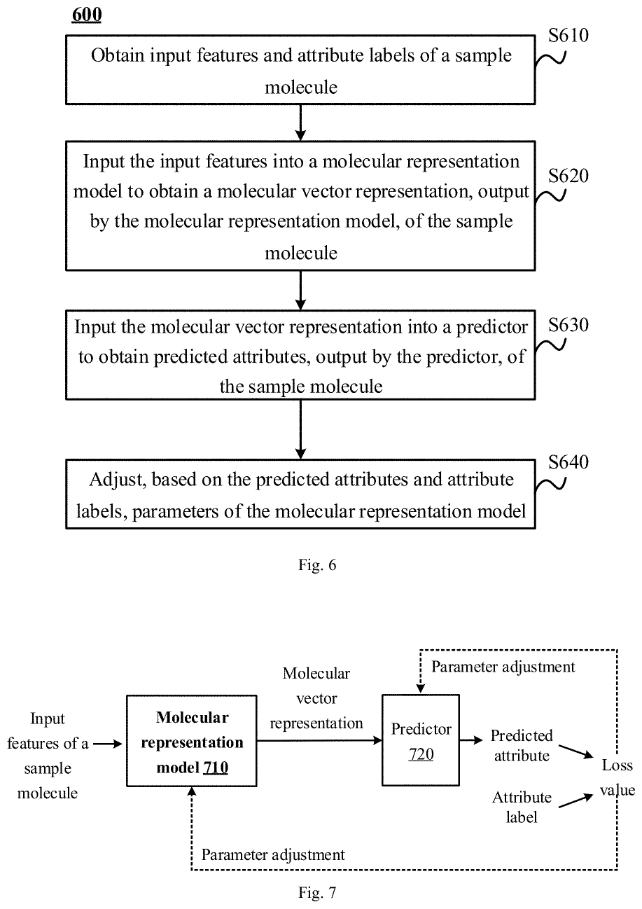

According to an embodiment of the present disclosure, a method for training a molecular representation model is further provided. FIG. 6 shows a flowchart of the method 600 for training the molecular representation model according to the embodiment of the present disclosure. The method 600 is generally executed at a server, and may also be executed at a client device. That is, an execution body of each step of the method 600 may be the server or the client device. As shown in FIG. 6, the method 600 includes steps S610-640.

In step S610, input features and attribute labels of a sample molecule are obtained. The sample molecule includes a plurality of atoms. The input features include a fully connected graph of the plurality of atoms, a plurality of atom vector representations, and a plurality of edge vector representations. The plurality of atom vector representations correspond to the plurality of atoms, respectively. The plurality of edge vector representations correspond to a plurality of edges included in the fully connected graph, respectively.

In step S620, the input features are input into the molecular representation model to obtain a molecular vector representation, output by the molecular representation model, of the sample molecule.

In step S630, the molecular vector representation is input into a predictor to obtain predicted attributes, output by the predictor, of the sample molecule.

In step S640, parameters of the molecular representation model are adjusted based on the predicted attributes and attribute labels.

According to the embodiment of the present disclosure, a trained molecular representation model may be obtained. The molecular representation model can generate the molecular vector representation of the molecule quickly and efficiently. In addition, due to joint training of the molecular representation model of the embodiment of the present disclosure and the predictor for molecular attributes, the molecular vector representation output by the molecular representation model can achieve a good attribute prediction effect, and accurate prediction of molecular attributes can be realized.

According to some embodiments, the predictor may be, for example, a feed forward neural network.

According to some embodiments, step S640 further includes: a loss value is calculated based on the predicted attributes and the attribute labels; and the parameters of the molecular representation model are adjusted based on the loss value. According to some embodiments, parameters of the predictor may also be adjusted based on the loss value.

A specific calculation manner of the loss value (that is, an expression of a loss function) may be determined according to a prediction task of the predictor. For example, when the prediction task is a classification task, loss functions such as cross entropy may be adopted; and when the prediction task is a regression task, loss functions such as a mean absolute error (MAE) and a mean square error (MSE) may be adopted.

It should be understood that the above steps S610-S640 may be performed repeatedly many times until a preset termination condition (for example, the loss value is less than a preset value, and the number of cycles reaches the preset maximum number of cycles) is met, so that the training process of the model ends, and the trained molecular representation model is obtained. According to some embodiments, a trained predictor may also be obtained.

FIG. 7 shows a schematic diagram of a training process of the molecular representation model according to some embodiments of the present disclosure. As shown in FIG. 7, the input features (including the fully connected graph, the plurality of atom vector representations and the plurality of edge vector representations) of the sample molecule are input into the molecular representation model 710, and the molecular representation model 710 outputs the molecular vector representation of the sample molecule. Then, the molecular vector representation is input into the predictor 720, and the predictor 720 outputs the predicted attributes of the sample molecule. Then, the loss values of the molecular representation model 710 and the predictor 720 are calculated based on the predicted attributes (predicted values) and the attribute labels (true values) of the sample molecule. Then, the parameters of the molecular representation model 710 and the predictor 720 are adjusted based on the loss values by using algorithms such as backpropagation.

According to some embodiments, the sample molecule further includes a plurality of chemical bonds among the plurality of atoms, and the plurality of edges at least include the plurality of chemical bonds. The method 600 further includes: atom feature information of each of the plurality of atoms and chemical bond feature information of each of the plurality of chemical bonds are obtained; a atom vector representation of each atom is generated at least based on the corresponding atom feature information; an edge vector representation of each chemical bond is generated at least based on the corresponding chemical bond feature information; and in response to determining that the number of the plurality of edges is greater than the number of the plurality of chemical bonds, an edge vector representation of each virtual edge is set to a preset value, where the virtual edge is any edge of the plurality of edges except the plurality of chemical bonds.

A generation manner of the atom vector representations, the edge vector representations of the chemical bonds, and the edge vector representations of the virtual edges may refer to the above description about step S130, which will not be repeated here.

According to some embodiments, the attribute labels and the predicted attributes each include at least one of: water solubility, toxicity, degree of matching with preset proteins, compound reactivity, stability, degradability, and energy.

The structure of the molecular representation model of the embodiment of the present disclosure will be described in detail below.

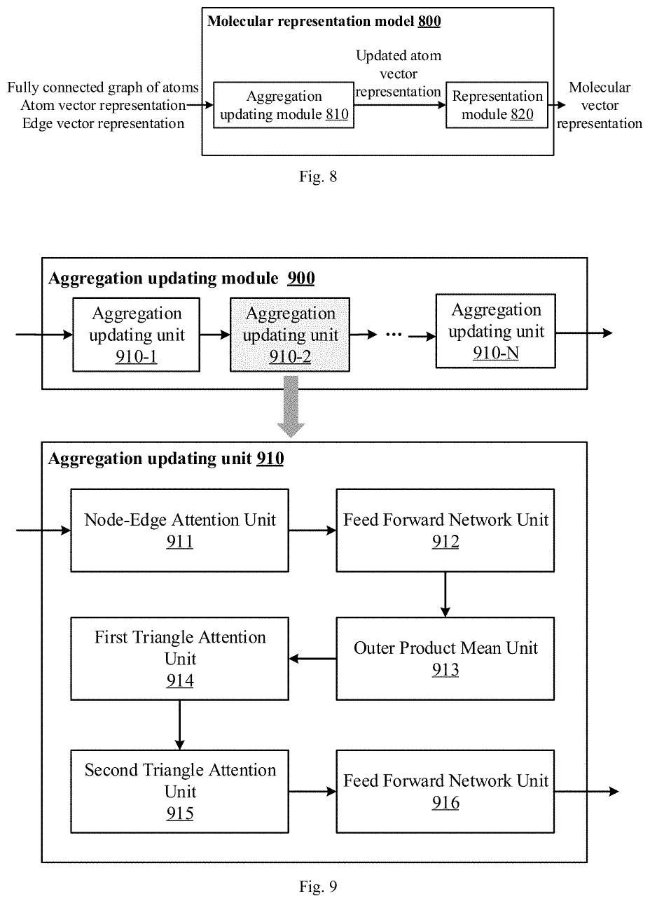

FIG. 8 shows a structural block diagram of a molecular representation model 800 according to some embodiments of the present disclosure. As shown in FIG. 8, the molecular representation model 800 includes an aggregation updating module 810 and a representation module 820. Correspondingly, the above step S620 further includes: the input features are input into the aggregation updating module 810 to obtain a plurality of updated atom vector representations output by the aggregation updating module 810, where the plurality of updated atom vector representations are obtained by performing, based on the fully connected graph, at least one aggregation on the plurality of atom vector representations and the plurality of edge vector representations; and the plurality of updated atom vector representations are input into the representation module 820 to obtain the molecular vector representation output by the representation module 820.

It should be understood that the aggregation updating module 810 may be configured to implement step S140 in the method 100 described with reference to FIG. 1; and the presentation module 820 may be configured to implement step S150 in the method 100 described with reference to FIG. 1.

FIG. 9 shows a structural block diagram of the aggregation updating module 900 according to the embodiment of the present disclosure. As shown in FIG. 9, the aggregation updating module 900 includes cascaded N (N≥1) aggregation updating units 910-1, 910-2, . . . , 910-N. The aggregation updating units 910 have the same structure. Each aggregation updating unit 910 is configured to perform one aggregation on the plurality of atom vector representations and the plurality of edge vector representations. For example, each aggregation updating unit 910 may be configured to implement steps S142-S146 in the method 100 described above.

As shown in FIG. 9, each aggregation updating unit 910 further includes a node-edge attention unit 911, a feed forward network unit 912, an outer product mean unit 913, a first triangle attention unit 914, a second triangle attention unit 915, and a feed forward network unit 916.

The node-edge attention unit 911 may be configured to update the atom vector representations. Specifically, the node-edge attention unit 911 performs the aggregation on the plurality of current atom vector representations and the plurality of current edge vector representations based on the attention mechanism to obtain the plurality of updated atom vector representations.

The node-edge attention unit 911 may be configured to implement step S142 in the method 100 described above.

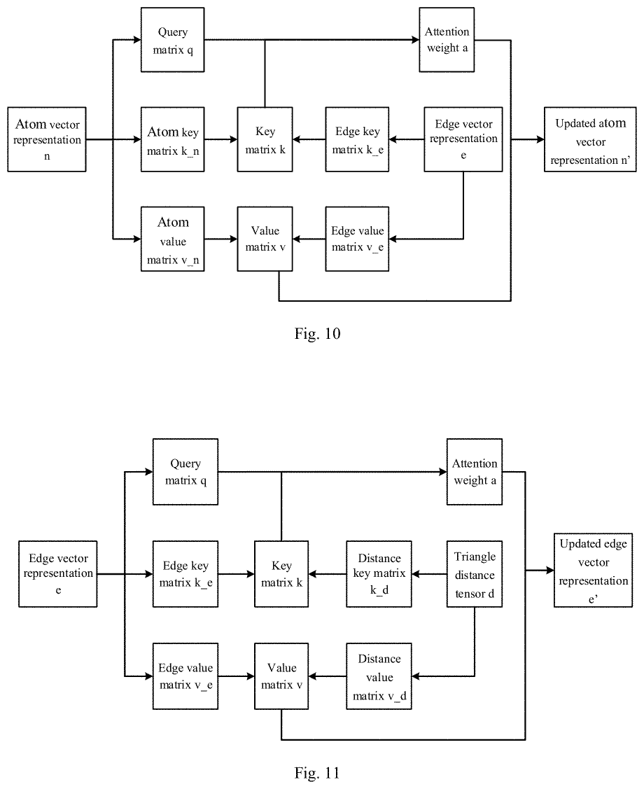

FIG. 10 shows a schematic diagram of a node-edge attention mechanism according to the embodiment of the present disclosure. The calculation process shown in FIG. 10 may be expressed as the following formulas (1)-(8):

$${q}^{T} , k\_n , v\_n = f ( n ) \qquad ( 1 )$$

$$k\_e = f ( e ) \qquad ( 2 )$$

$$k = k\_n + k\_e \qquad ( 3 )$$

$$v\_e = f ( e ) \qquad ( 4 )$$

$$v = v\_n + v\_e \qquad ( 5 )$$

$$g = g ( f ( n ) ) \qquad ( 6 )$$

$$a = softmax ( \frac{1}{\sqrt{c}} {q}^{T} k ) \qquad ( 7 )$$

$${n}^{′} = g ⊙ \sum av \qquad ( 8 )$$

In the above formulas (1)-(8), q, k, and v represent a query matrix, a key matrix, and a value matrix, respectively. T represents transposition. n represents the plurality of current atom vector representations. e represents the plurality of current edge vector representations. f and g are functional layer processing functions, such as the linear transformation function and the sigmoid activation function. a represents an attention weight. c represents a dimension of the atom vector representations. ⊙ represents an element-wise product, also known as a Hadamard product. n′ represents the plurality of updated edge vector representations.

The feed forward network unit 912 is configured to perform linear transformation on the plurality of updated atom vector representations output by the node-edge attention unit 911, so as to improve the fitting capacity of the model.

The outer product mean unit 913 is configured to add the plurality of updated atom vector representations and the plurality of current edge vector representations, so as to realize supplementation and augmentation of edge information. That is, the current edge vector representation of any edge of the plurality of edges is updated based on the updated atom vector representations of two atoms connected by the edge to obtain a first edge vector representation of the edge.

Specifically, the outer product mean unit 913 determines a vector representation variation of any edge of the plurality of edges based on the updated atom vector representations of the two atoms connected by the edge, and adds the current edge vector representation and the vector representation variation to obtain the first edge vector representation of the edge.

The outer product mean unit 913 may be configured to implement steps S144, S1442 and S1444 in the method 100 described above.

The first triangle attention unit 914 and the second triangle attention unit 915 are configured to implement an aggregation of the edge vector representations based on adjacent edge pairs. Since an edge and an adjacent edge pair can form a triangle, an edge attention unit can also be called a triangle attention unit. Specifically, the first triangle attention unit 914 performs aggregation on any edge of the plurality of edges and the first edge vector representation of each first adjacent edge in at least one adjacent edge pair based on the attention mechanism to obtain a second edge vector representation of the edge. The second triangle attention unit 915 performs aggregation on the edge and a second edge vector representation of each second adjacent edge in the at least one adjacent edge pair based on the attention mechanism to obtain the updated edge vector representation of the edge.

The first triangle attention unit 914 and the second triangle attention unit 915 are jointly configured to implement steps S146 and S1464 in the method 100 described above. More specifically, the first triangle attention unit 914 and the second triangle attention unit 915 may be configured to implement steps S14642 and S14644 in the method 100 described above respectively.

FIG. 11 shows a schematic diagram of an edge attention (triangle attention) mechanism according to some embodiments of the present disclosure. The first triangle attention unit 914 and the second triangle attention unit 915 may adopt a calculation process shown in FIG. 11. In order to reflect the generality of the first triangle attention unit 914 and the second triangle attention unit 915 in FIG. 11, the subscripts i, j, and k of each parameter are not indicated in FIG. 11.

Referring to FIG. 11, the calculation process of the first triangle attention unit 914 can be expressed as the following equations (9)-(15):

$${q}_{ij}^{T} , {k\_e}_{ij} , {v\_e}_{ij} = f ( {e}_{ij} ) \qquad ( 9 )$$

$${k\_d}_{ij} , {v\_d}_{ij} = f ( {d}_{ij} ) \qquad ( 10 )$$

$${k}_{i k} = {k\_e}_{ij} + {k\_d}_{ij} \qquad ( 11 )$$

$${v}_{i k} = {v\_e}_{ij} + {v\_d}_{ij} \qquad ( 12 )$$

$${a}_{ijk} = {softmax}_{k} ( \frac{1}{\sqrt{c}} {q}_{ij}^{T} {k}_{i k} ) \qquad ( 13 )$$

$${g}_{ij} = g ( f ( {e}_{ij} ) ) \qquad ( 14 )$$

$${e}_{ij}^{′} = {g}_{ij} ⊙ {\sum }_{k} {a}_{ijk} {v}_{ik} \qquad ( 15 )$$

In the above formulas (9)-(15), q, k, and v represent a query matrix, a key matrix, and a value matrix, respectively. T represents transposition. eᵢⱼ represents a first edge vector representation of an edge ij (that is, an edge between a atom i and a atom j). eᵢⱼ′ represents a second edge vector representation of the edge ij. f and g are functional layer processing functions, such as the linear transformation function (Linear) and the sigmoid activation function. aᵢⱼₖ represents an attention weight of an edge ik. c represents a dimension of the edge vector representations. ⊙ represents an element-wise product, also known as a Hadamard product.

d is a triangle distance tensor. d is a four-dimensional tensor, where the first three dimensions represent the three atoms i, j, and k of the triangle, respectively, and the fourth dimension represents the shortest chemical bond distance between every two of the atoms i, j, and k. dᵢⱼ represents an element, with a first dimension being i and a second dimension being j, in the tensor d.

Referring to FIG. 11, the calculation process of the second triangle attention unit 915 may be expressed as the following equations (16)-(22):

$${q}_{ij}^{T} , {k\_e}_{ij} , {v\_e}_{ij} = f ( {e}_{ij} ) \qquad ( 16 )$$

$${k\_d}_{ij} , {v\_d}_{ij} = f ( {d}_{ij} ) \qquad ( 17 )$$

$${k}_{kj} = {k\_e}_{ij} + {k\_d}_{ij} \qquad ( 18 )$$

$${v}_{kj} = {v\_e}_{ij} + {v\_d}_{ij} \qquad ( 19 )$$

$${a}_{ijk} = {softmax}_{k} ( \frac{1}{\sqrt{c}} {q}_{ij}^{T} {k}_{kj} ) \qquad ( 20 )$$

$${g}_{ij} = g ( f ( {e}_{ij} ) ) \qquad ( 21 )$$

$${e}_{ij}^{′} = {g}_{ij} ⊙ {\sum }_{k} {a}_{ijk} {v}_{kj} \qquad ( 22 )$$

In the above formulas (16)-(22), q, k, and v represent a query matrix, a key matrix, and a value matrix, respectively. T represents transposition. eᵢⱼ represents a second edge vector representation of the edge ij (that is, the edge between the atom i and the atom j). eᵢⱼ′ represents an updated edge vector representation of the edge ij. f and g are functional layer processing functions, such as the linear transformation function and the sigmoid activation function. aᵢⱼₖ represents an attention weight of an edge kj. c represents a dimension of the edge vector representations. ⊙ represents an element-wise product, also known as a Hadamard product.

d is a triangle distance tensor. d is a four-dimensional tensor, where the first three dimensions represent the three atoms i, j, and k of the triangle, respectively, and the fourth dimension represents the shortest chemical bond distance between every two of the atoms i, j, and k. dᵢⱼ represents an element, with a first dimension being i and a second dimension being j, in the tensor d.

The feed forward network unit 916 is configured to perform linear transformation on the plurality of updated edge vector representations output by the second triangle attention unit 915, so as to improve the fitting capacity of the model.

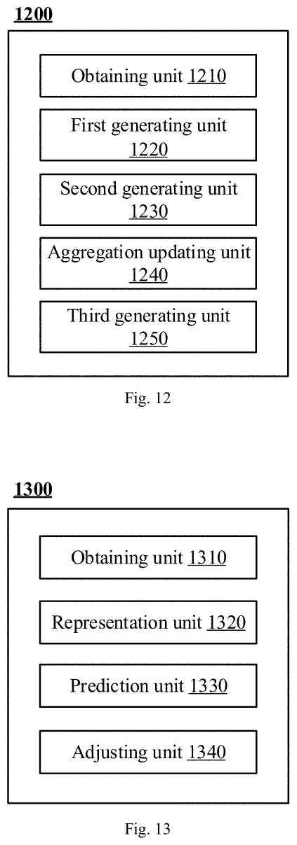

According to an embodiment of the present disclosure, a molecular representation apparatus is further provided. FIG. 12 shows a structural block diagram of the molecular representation apparatus 1200 according to the embodiment of the present disclosure. As shown in FIG. 12, the apparatus 1200 includes:

an obtaining unit 1210, configured to obtain feature information of a molecule to be represented, where the molecule includes a plurality of atoms;

a first generating unit 1220, configured to generate a fully connected graph of the plurality of atoms, where the fully connected graph includes a plurality of edges;

a second generating unit 1230, configured to generate, based on the feature information, a plurality of atom vector representations and a plurality of edge vector representations, where the plurality of atom vector representations correspond to the plurality of atoms, respectively, and the plurality of edge vector representations correspond to the plurality of edges, respectively;

an aggregation updating unit 1240, configured to perform, based on the fully connected graph, at least one aggregation on the plurality of atom vector representations and the plurality of edge vector representations to obtain a plurality of updated atom vector representations; and

a third generating unit 1250, configured to generate, based on the plurality of updated atom vector representations, a molecular vector representation of the molecule.

Attributes of the molecule are essentially a result of interaction between the atoms, and edges between the atoms can express the connectivity and interaction between the atoms. According to the embodiment of the present disclosure, by constructing the fully connected graph of the atoms and performing aggregation on the atom vector representations and the edge vector representations, atom information and edge information can be fully interacted, thereby obtaining the more comprehensive and accurate molecular vector representation.

The molecular vector representation of the embodiment of the present disclosure can fully and accurately express the properties of the molecule. Further, by predicting the attributes of the molecules according to the molecular vector representation of the embodiment of the present disclosure, the accuracy of molecular attribute prediction can be improved, thereby greatly improving the efficiency of drug research and development.

According to an embodiment of the present disclosure, an apparatus for training a molecular representation model is further provided. FIG. 13 shows a structural block diagram of the apparatus 1300 for training the molecular representation model according to the embodiment of the present disclosure. As shown in FIG. 13, the apparatus 1300 includes:

an obtaining unit 1310, configured to obtain input features and attribute labels of a sample molecule, wherein the sample molecule includes a plurality of atoms, the input features include a fully connected graph of the plurality of atoms, a plurality of atom vector representations, and a plurality of edge vector representations, the plurality of atom vector representations correspond to the plurality of atoms, respectively, and the plurality of edge vector representations correspond to a plurality of edges included in the fully connected graph, respectively;

a representation unit 1320, configured to input the input features into the molecular representation model to obtain a molecular vector representation, output by the molecular representation model, of the sample molecule;

a prediction unit 1330, configured to input the molecular vector representation into a predictor to obtain predicted attributes, output by the predictor, of the sample molecule; and

an adjusting unit 1340, configured to adjust, based on the predicted attributes and the attribute labels, parameters of the molecular representation model.

According to the embodiment of the present disclosure, a trained molecular representation model may be obtained. The molecular representation model can generate the molecular vector representation of the molecule quickly and efficiently. In addition, due to joint training of the molecular representation model of the embodiment of the present disclosure and the predictor for molecular attributes, the molecular vector representation output by the molecular representation model can achieve a good attribute prediction effect, and accurate prediction of molecular attributes can be realized.

It should be understood that the units of the apparatus 1200 shown in FIG. 12 may correspond to the steps in the method 100 described with reference to FIG. 1, and the units of the apparatus 1300 shown in FIG. 13 may correspond to the steps in the method 600 described with reference to FIG. 6. Thus, the operations, features and advantages described above for the method 100 are equally applicable to the apparatus 1200 and the units thereof, and the operations, features and advantages described above for the method 600 are also applicable to the apparatus 1300 and the units thereof. For the sake of conciseness, certain operations, features, and advantages are not repeated here.

Although specific functions are discussed above with reference to specific units, it should be noted that the functions of the units discussed herein may be divided into a plurality of elements, and/or at least some of the functions of the plurality of units may be combined into a single unit. For example, the first generating unit 1220 and the second generating unit 1230 described above may be combined into a single unit in some embodiments.

It should also be understood that various techniques can be described herein in the general context of software/hardware elements or program units. The various units described above with respect to FIG. 12 and FIG. 13 may be implemented in hardware or in hardware in combination with software and/or firmware. For example, the units may be implemented as computer program codes/instructions configured to be executed in one or more processors and stored in a computer-readable storage medium. Alternatively, the units may be implemented as hardware logic/circuitry. For example, in some embodiments, one or more of the units 1210-1340 may be implemented jointly in a system on chip (SoC). The SoC may include an integrated circuit chip which includes a processor (for example, a central processing unit (CPU), a microcontroller, a microprocessor, a digital signal processor (DSP)), a memory, one or more communication interfaces, and/or one or more components of other circuits, and may optionally execute received program codes and/or include embedded firmware to perform functions.

According to an embodiment of the present disclosure, an electronic device is provided, and includes: at least one processor; and a memory in communication connection with the at least one processor. The memory stores instructions capable of being executed by the at least one processor, and the instructions are executed by the at least one processor so that the at least one processor can execute the molecular representation method and/or the method for training the molecular representation model according to the embodiments of the present disclosure.

According to one aspect of the present disclosure, a non-transitory computer readable storage medium storing computer instructions is provided. The computer instructions are configured to enable a computer to execute the molecular representation method and/or the method for training the molecular representation model according to the embodiments of the present disclosure.

According to one aspect of the present disclosure, a computer program product is provided, and includes a computer program. The computer program, when executed by a processor, implements the molecular representation method and/or the method for training the molecular representation model according to the embodiments of the present disclosure.

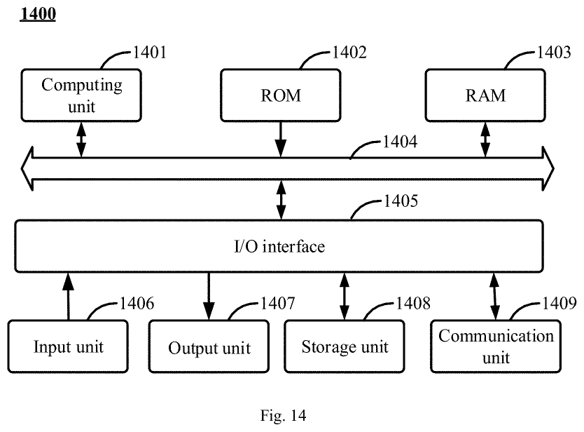

Referring to FIG. 14, a structural block diagram of an electronic device 1400 that may serve as a server or a client of the present disclosure will now be described, and the electronic device is an example of a hardware device that may be applied to various aspects of the present disclosure. The electronic device is intended to represent various forms of digital electronic computer devices, such as a laptop computer, a desktop computer, a workstation, a personal digital assistant, a server, a blade server, a mainframe computer, and other suitable computers. The electronic device may further represent various forms of mobile apparatuses, such as a personal digital assistant, a cell phone, a smart phone, a wearable device and other similar computing apparatuses. The components shown herein, their connections and relationships, and their functions are merely used as examples, and are not intended to limit the implementations of the present disclosure described and/or required herein.

As shown in FIG. 14, the electronic device 1400 includes a computing unit 1401 that may perform various appropriate actions and processing according to computer programs stored in a read-only memory (ROM) 1402 or computer programs loaded from a storage unit 1408 into a random access memory (RAM) 1403. Various programs and data required for operations of the device 1400 may further be stored in the RAM 1403. The computing unit 1401, the ROM 1402 and the RAM 1403 are connected to each other via a bus 1404. An input/output (I/O) interface 1405 is also connected to the bus 1404.

A plurality of components in the electronic device 1400 are connected to the I/O interface 1405, including: an input unit 1406, an output unit 1407, a storage unit 1408, and a communication unit 1409. The input unit 1406 may be any type of device capable of inputting information to the device 1400. The input unit 1406 may receive input digital or character information and generate key signal input related to user settings and/or function control of the electronic device, and may include but not limited to a mouse, a keyboard, a touch screen, a trackpad, a trackball, a joystick, a microphone and/or a remote control. The output unit 1407 may be any type of device capable of presenting information, and may include but not limited to a display, a speaker, a video/audio output terminal, a vibrator and/or a printer. The storage unit 1408 may include, but not limited to, a magnetic disk and a compact disk. The communication unit 1409 allows the device 1400 to exchange information/data with other devices via computer networks such as the Internet and/or various telecommunication networks, and may include, but not limited to, a modem, a network card, an infrared communication device, a wireless communication transceiver and/or a chipset, such as a Bluetooth™ device, a 802.11 device, a Wi-Fi device, a WiMax device, a cellular communication device and/or the like.

The computing unit 1401 may be various general-purpose and/or special-purpose processing components with processing and computing capabilities. Some examples of the computing unit 1401 include, but not limited to, a central processing unit (CPU), a graphics processing unit (GPU), various dedicated artificial intelligence (AI) computing chips, various computing units running machine learning model algorithms, a digital signal processor (DSP), and any appropriate processor, controller, microcontroller, etc. The computing unit 1401 performs various methods and processing described above, such as the 100 and the method 600. For example, in some embodiments, the method 100 and/or the method 600 may be implemented as a computer software program that is tangibly included in a machine-readable medium such as the storage unit 1408. In some embodiments, part or all of the computer programs may be loaded and/or installed onto the device 1400 via the ROM 1402 and/or the communication unit 1409. When the computer programs are loaded into the RAM 1403 and executed by the computing unit 1401, one or more steps of the method 100 and/or the method 600 described above may be performed. Alternatively, in other embodiments, the computing unit 1401 may be configured to perform the method 100 and/or the method 600 in any other suitable manner (for example, by means of firmware).

Various implementations of the systems and technologies described above in this paper may be implemented in a digital electronic circuit system, an integrated circuit system, a field programmable gate array (FPGA), an application specific integrated circuit (ASIC), an application specific standard part (ASSP), a system on chip (SOC), a complex programmable logic device (CPLD), computer hardware, firmware, software and/or their combinations. These various implementations may include: being implemented in one or more computer programs, wherein the one or more computer programs may be executed and/or interpreted on a programmable system including at least one programmable processor, and the programmable processor may be a special-purpose or general-purpose programmable processor, and may receive data and instructions from a storage system, at least one input apparatus, and at least one output apparatus, and transmit the data and the instructions to the storage system, the at least one input apparatus, and the at least one output apparatus.

Program codes for implementing the methods of the present disclosure may be written in any combination of one or more programming languages. These program codes may be provided to processors or controllers of a general-purpose computer, a special-purpose computer or other programmable data processing apparatuses, so that when executed by the processors or controllers, the program codes enable the functions/operations specified in the flow diagrams and/or block diagrams to be implemented. The program codes may be executed completely on a machine, partially on the machine, partially on the machine and partially on a remote machine as a separate software package, or completely on the remote machine or server.

In the context of the present disclosure, a machine readable medium may be a tangible medium that may contain or store a program for use by or in connection with an instruction execution system, apparatus or device. The machine readable medium may be a machine readable signal medium or a machine readable storage medium. The machine readable medium may include but not limited to an electronic, magnetic, optical, electromagnetic, infrared, or semiconductor system, apparatus or device, or any suitable combination of the above contents. More specific examples of the machine readable storage medium will include electrical connections based on one or more lines, a portable computer disk, a hard disk, a random access memory (RAM), a read only memory (ROM), an erasable programmable read only memory (EPROM or flash memory), an optical fiber, a portable compact disk read only memory (CD-ROM), an optical storage device, a magnetic storage device, or any suitable combination of the above contents.

In order to provide interactions with users, the systems and techniques described herein may be implemented on a computer, and the computer has: a display apparatus for displaying information to the users (e.g., a CRT (cathode ray tube) or LCD (liquid crystal display) monitor); and a keyboard and a pointing device (e.g., a mouse or trackball), through which the users may provide input to the computer. Other types of apparatuses may further be used to provide interactions with users; for example, feedback provided to the users may be any form of sensory feedback (e.g., visual feedback, auditory feedback, or tactile feedback); an input from the users may be received in any form (including acoustic input, voice input or tactile input).

The systems and techniques described herein may be implemented in a computing system including background components (e.g., as a data server), or a computing system including middleware components (e.g., an application server) or a computing system including front-end components (e.g., a user computer with a graphical user interface or a web browser through which a user may interact with the implementations of the systems and technologies described herein), or a computing system including any combination of such background components, middleware components, or front-end components. The components of the system may be interconnected by digital data communication (e.g., a communication network) in any form or medium. Examples of the communication network include: a local area network (LAN), a wide area network (WAN) and the Internet.

A computer system may include a client and a server. The client and the server are generally far away from each other and usually interact via a communication network. The relationship between the client and the server is generated by computer programs running on the corresponding computer and having a client-server relationship with each other. The server may be a cloud server, or a server of a distributed system, or a server combined with a block chain.

It should be understood that the various forms of processes shown above may be used to reorder, add, or delete steps. For example, the steps recorded in the present disclosure may be performed in parallel, sequentially or in different orders, as long as the desired results of the technical solution disclosed by the present disclosure can be achieved, which is not limited herein.

Although the embodiments or examples of the present disclosure have been described with reference to the accompanying drawings, it should be understood that the above methods, systems and devices are only embodiments or examples, and the scope of the present disclosure is not limited by these embodiments or examples, but only by the authorized claims and their equivalent scope. Various elements in the embodiments or examples may be omitted or replaced by their equivalent elements. In addition, the steps may be performed in an order different from that described in the present disclosure. Further, various elements in the embodiments or examples may be combined in various ways. It is important that as technology evolves, many of the elements described herein may be replaced by equivalent elements that appear after the present disclosure.

1. A computer-implemented method, comprising:
obtaining a feature information of a molecule, wherein the molecule comprises a plurality of atoms;
generating a fully connected graph of the plurality of atoms, wherein the fully connected graph comprises a plurality of edges;
generating, based on the feature information, a plurality of atom vector representations and a plurality of edge vector representations, wherein the plurality of atom vector representations correspond to the plurality of atoms respectively, and the plurality of edge vector representations correspond to the plurality of edges respectively;
performing, based on the fully connected graph, at least one aggregation on the plurality of atom vector representations and the plurality of edge vector representations to obtain a plurality of updated atom vector representations; and
generating, based on the plurality of updated atom vector representations, a molecular vector representation of the molecule.

2. The method according to claim 1, wherein the molecule further comprises a plurality of chemical bonds among the plurality of atoms, wherein the feature information comprises an atom feature information of each of the plurality of atoms and a chemical bond feature information of each of the plurality of chemical bonds, wherein the plurality of edges comprise at least the plurality of chemical bonds, and wherein the generating, based on the feature information, the plurality of atom vector representations and the plurality of edge vector representations comprises:
generating, for each atom of the plurality of atoms, an atom vector representation of the atom at least based on a corresponding atom feature information of the atom;
generating, for each chemical bond of the plurality of chemical bonds, an edge vector representation of the chemical bond at least based on a corresponding chemical bond feature information of the chemical bond; and
setting, in response to determining that a number of the plurality of edges is greater than a number of the plurality of chemical bonds, an edge vector representation of each virtual edge to a preset value, wherein the virtual edge is any edge of the plurality of edges except the plurality of chemical bonds.

3. The method according to claim 1, wherein each aggregation of the at least one aggregation comprises:
performing, for any atom of the plurality of atoms, an aggregation on a plurality of current atom vector representations and a plurality of current edge vector representations to obtain an updated atom vector representation of the atom based on an attention mechanism; and
for any edge of the plurality of edges:
updating, based on the updated atom vector representation of each of two atoms connected by the edge, a current edge vector representation of the edge to obtain a first edge vector representation of the edge; and
performing, based on the attention mechanism, aggregation on a plurality of first edge vector representations of the plurality of edges to obtain an updated edge vector representation of the edge.

4. The method according to claim 3, wherein the updating, based on the updated atom vector representation of each of two atoms connected by the edge, the current edge vector representation of the edge to obtain the first edge vector representation of the edge comprises:
determining, based on the updated atom vector representation of each of the two atoms connected by the edge, a vector representation variation of the edge; and
adding the current edge vector representation and the vector representation variation of the edge to obtain the first edge vector representation of the edge.

5. The method according to claim 3, wherein the performing, based on the attention mechanism, aggregation on the plurality of first edge vector representations of the plurality of edges to obtain the updated edge vector representation of the edge comprises:
determining at least one adjacent edge pair of the edge, wherein each adjacent edge pair of the at least one adjacent edge pair comprises two adjacent edges of the edge, and the two adjacent edges are connected with the edge to form a triangle; and
performing, based on the attention mechanism, aggregation on the first edge vector representation of each of the edge and each adjacent edge in the at least one adjacent edge pair to obtain the updated edge vector representation of the edge.

6. The method according to claim 5, wherein the two adjacent edges of each adjacent edge pair of the at least one adjacent edge pair comprise a first adjacent edge connected to a first end point of the edge and a second adjacent edge connected to a second end point of the edge, and wherein the performing, based on the attention mechanism, aggregation on the first edge vector representation of each of the edge and each adjacent edge in the at least one adjacent edge pair to obtain the updated edge vector representation of the edge comprises:
performing, based on the attention mechanism, aggregation on the edge and a first edge vector representation of each first adjacent edge in the at least one adjacent edge pair to obtain a second edge vector representation of the edge; and
performing, based on the attention mechanism, aggregation on the edge and a second edge vector representation of each second adjacent edge in the at least one adjacent edge pair to obtain the updated edge vector representation of the edge.

7. The method according to claim 5, wherein an attention weight of the edge and each adjacent edge in the at least one adjacent edge pair is determined at least based on a shortest chemical bond distance between two atoms corresponding to the edge.

8. The method according to claim 1, further comprising:
predicting, based on the molecular vector representation, at least one attribute of the molecule.

9. The method according to claim 8, wherein the at least one attribute comprises at least one of:
a water solubility, a toxicity, a degree of matching with preset proteins, a compound reactivity, a stability, a degradability, and an energy.

10. The method according to claim 1, wherein the performing, based on the fully connected graph, at least one aggregation on the plurality of atom vector representations and the plurality of edge vector representations to obtain a plurality of updated atom vector representations comprises:
inputting the fully connected graph, the plurality of atom vector representations and the plurality of edge vector representations into an aggregation updating module of a trained molecular representation model to obtain the plurality of updated atom vector representations output by the aggregation updating module, and wherein
the generating, based on the plurality of updated atom vector representations, the molecular vector representation of the molecule comprises:
inputting the plurality of updated atom vector representations into a representation module of the trained molecular representation model to obtain the molecular vector representation, output by the representation module, of the molecule.

11. The method according to claim 10, wherein the trained molecular representation model is trained based on operations comprising:
obtaining input features and at least one attribute label of a sample molecule, wherein the sample molecule comprises a plurality of first atoms, wherein the input features comprise a first fully connected graph of the plurality of first atoms, a plurality of first atom vector representations, and a plurality of edge vector representations of the sample molecule, wherein the plurality of first atom vector representations correspond to the plurality of first atoms, respectively, and wherein the plurality of edge vector representations of the sample molecule correspond to a plurality of edges comprised in the first fully connected graph, respectively;
inputting the input features into a molecular representation model to obtain a first molecular vector representation, output by the molecular representation model, of the sample molecule;
inputting the first molecular vector representation into a predictor to obtain at least one predicted attribute, output by the predictor, of the sample molecule; and
adjusting, based on the at least one predicted attribute and the at least one attribute label, parameters of the molecular representation model to obtain the trained molecular representation model.

12. The method according to claim 11, wherein the sample molecule further comprises a plurality of first chemical bonds among the plurality of first atoms; the plurality of edges comprised in the first fully connected graph comprise at least the plurality of first chemical bonds; and the method further comprises:
obtaining a first atom feature information of each of the plurality of first atoms and a first chemical bond feature information of each of the plurality of first chemical bonds;
generating, for each first atom of the plurality of first atoms, a first atom vector representation of the first atom at least based on a corresponding first atom feature information of the first atom;
generating, for each first chemical bond of the plurality of first chemical bonds, an edge vector representation of the first chemical bond at least based on a corresponding first chemical bond feature information of the first chemical bond; and
setting, in response to determining that a number of the plurality of edges comprised in the first fully connected graph is greater than a number of the plurality of first chemical bonds, an edge vector representation of each first virtual edge to a first preset value, wherein the first virtual edge is any edge of the plurality of edges comprised in the first fully connected graph except the plurality of first chemical bonds.

13. The method according to claim 11, wherein the inputting the input features into the molecular representation model to obtain the first molecular vector representation, output by the molecular representation model, of the sample molecule comprises:
inputting the input features into the aggregation updating module to obtain a plurality of updated first atom vector representations output by the aggregation updating module, wherein the plurality of updated first atom vector representations are obtained by performing, based on the first fully connected graph, at least one aggregation on the plurality of first atom vector representations and the plurality of edge vector representations of the sample molecule; and
inputting the plurality of updated first atom vector representations into the representation module to obtain the first molecular vector representation output by the representation module.

14. The method according to claim 11, wherein the at least one attribute labels and the at least one predicted attributes respectively comprise at least one of:
a water solubility, a toxicity, a degree of matching with preset proteins, a compound reactivity, a stability, a degradability, and an energy.

15. An electronic device, comprising:
one or more processors; and
a memory storing one or more programs configured to be executed by the one or more processors, the one or more programs comprising instructions for performing operations comprising:
obtaining a feature information of a molecule, wherein the molecule comprises a plurality of atoms;
generating a fully connected graph of the plurality of atoms, wherein the fully connected graph comprises a plurality of edges;
generating, based on the feature information, a plurality of atom vector representations and a plurality of edge vector representations, wherein the plurality of atom vector representations correspond to the plurality of atoms respectively, and the plurality of edge vector representations correspond to the plurality of edges respectively;
performing, based on the fully connected graph, at least one aggregation on the plurality of atom vector representations and the plurality of edge vector representations to obtain a plurality of updated atom vector representations; and
generating, based on the plurality of updated atom vector representations, a molecular vector representation of the molecule.

16. The electronic device according to claim 15, wherein the molecule further comprises a plurality of chemical bonds among the plurality of atoms, wherein the feature information comprises an atom feature information of each of the plurality of atoms and a chemical bond feature information of each of the plurality of chemical bonds, wherein the plurality of edges comprise at least the plurality of chemical bonds, and wherein the generating, based on the feature information, the plurality of atom vector representations and the plurality of edge vector representations comprises:
generating, for each atom of the plurality of atoms, an atom vector representation of the atom at least based on a corresponding atom feature information of the atom;
generating, for each chemical bond of the plurality of chemical bonds, an edge vector representation of the chemical bond at least based on a corresponding chemical bond feature information of the chemical bond; and
setting, in response to determining that a number of the plurality of edges is greater than a number of the plurality of chemical bonds, an edge vector representation of each virtual edge to a preset value, wherein the virtual edge is any edge of the plurality of edges except the plurality of chemical bonds.

17. The electronic device according to claim 15, wherein the performing, based on the fully connected graph, at least one aggregation on the plurality of atom vector representations and the plurality of edge vector representations to obtain a plurality of updated atom vector representations comprises:
inputting the fully connected graph, the plurality of atom vector representations and the plurality of edge vector representations into an aggregation updating module of a trained molecular representation model to obtain the plurality of updated atom vector representations output by the aggregation updating module, and wherein
the generating, based on the plurality of updated atom vector representations, the molecular vector representation of the molecule comprises:
inputting the plurality of updated atom vector representations into a representation module of the trained molecular representation model to obtain the molecular vector representation, output by the representation module, of the molecule.

18. A non-transitory computer-readable storage medium storing one or more programs comprising instructions that, when executed by one or more processors of a computing device, cause the computing device to perform operations comprising:
obtaining a feature information of a molecule, wherein the molecule comprises a plurality of atoms;
generating a fully connected graph of the plurality of atoms, wherein the fully connected graph comprises a plurality of edges;
generating, based on the feature information, a plurality of atom vector representations and a plurality of edge vector representations, wherein the plurality of atom vector representations correspond to the plurality of atoms respectively, and the plurality of edge vector representations correspond to the plurality of edges respectively;
performing, based on the fully connected graph, at least one aggregation on the plurality of atom vector representations and the plurality of edge vector representations to obtain a plurality of updated atom vector representations; and
generating, based on the plurality of updated atom vector representations, a molecular vector representation of the molecule.

19. The computer-readable storage medium of claim 18, wherein the molecule further comprises a plurality of chemical bonds among the plurality of atoms, wherein the feature information comprises an atom feature information of each of the plurality of atoms and a chemical bond feature information of each of the plurality of chemical bonds, wherein and the plurality of edges comprise at least the plurality of chemical bonds, and wherein the generating, based on the feature information, the plurality of atom vector representations and the plurality of edge vector representations comprises:
generating, for each atom of the plurality of atoms, an atom vector representation of the atom at least based on a corresponding atom feature information of the atom;
generating, for each chemical bond of the plurality of chemical bonds, an edge vector representation of the chemical bond at least based on a corresponding chemical bond feature information of the chemical bond; and
setting, in response to determining that a number of the plurality of edges is greater than a number of the plurality of chemical bonds, an edge vector representation of each virtual edge to a preset value, wherein the virtual edge is any edge of the plurality of edges except the plurality of chemical bonds.

20. The computer-readable storage medium of claim 18, wherein the performing, based on the fully connected graph, at least one aggregation on the plurality of atom vector representations and the plurality of edge vector representations to obtain a plurality of updated atom vector representations comprises:
inputting the fully connected graph, the plurality of atom vector representations and the plurality of edge vector representations into an aggregation updating module of a trained molecular representation model to obtain the plurality of updated atom vector representations output by the aggregation updating module, and wherein
the generating, based on the plurality of updated atom vector representations, the molecular vector representation of the molecule comprises:
inputting the plurality of updated atom vector representations into a representation module of the trained molecular representation model to obtain the molecular vector representation, output by the representation module, of the molecule.
